(19) United States
(12) Patent Application Publication
(10) Pub. No.: US 2009/0248065 A1
(43) Pub. Date: Oct. 1, 2009

(54) HEMOSTATIC DEVICE SEALING DAMAGED SITE WITH VACUUM

(75) Inventors: Yuichiro SATO, Tokyo (JP); Kazuyuki SUZUKI, Tokyo (JP); Yoshiro YAMAMOTO, Tokyo (JP)

Correspondence Address: CANTOR COLBURN, LLP, 20 Church Street, 22nd Floor, Hartford, CT, 06103, US

(73) Assignee: MIWATEC CO., LTD., Tokyo (JP)

(21) Appl. No.: 12/412,043

(22) Filed: Mar. 26, 2009

(30) Foreign Application Priority Data
Mar. 26, 2008 (JP) 2008-080633

(51) Int. Cl. A61B 17/08

(52) U.S. Cl. 606/216; 606/213

(57) ABSTRACT

A hemostatic device includes a hemostatic head that includes a peripheral end surface formed at a distal end of the hemostatic head, the peripheral end surface extending along an outer edge region of the distal end in a circumferential direction of the hemostatic head and having suction ports formed on the peripheral end surface, a recess that is open at the distal end and that is surrounded by the peripheral end surface, a plurality of suction channels that are formed inside the hemostatic head, the suction channels communicating with the respective suction ports. The device also includes a suction device for applying suction pressure to the suction ports via the suction channels, the suction device being coupled to the suction channels.

The present application is based on, and claims priority from, J.P. Application No. 2008-080633, filed on Mar. 26, 2008, the disclosure of which is hereby incorporated by reference herein in its entirety.

### BACKGROUND OF THE INVENTION

1. Field of the Invention

The present invention relates to a hemostatic device for stanching the bleeding site of a patient and a stanching method using the same.

2. Description of the Related Art

A mistake that a doctor can make during an operation, or a serious injury that a patient has sustained, may damage the artery or vein of the patient, causing heavy bleeding. In this case, the damaged site is generally sutured for hemostatic treatment. However, before stanching of the damaged site is completed, the patient may lapse into a shock state, or, in the worst case, even into a critical state because of the heavy bleeding. This means that the patient's life, otherwise lost due to the heavy bleeding, will be saved if the damaged site of a patient suffering heavy bleeding is immediately stanched.

In conventional medical practice, hemostatic treatment is performed as follows. First, in order to make a damaged site visible, blood that stays at the damaged site is sucked out and removed by mean of a suction device. Alternatively, blood that stays at the damaged site is removed by using gauze that soaks up blood. The damaged bleeding site is thus identified and hemostatic treatment is performed, for example, by suturing the site. The above-described hemostatic treatment is a commonly used medical technique in medical practice.

However, since suturing the damaged bleeding site requires a certain amount of time, a heavy loss of blood may occur during the suturing treatment. Furthermore, blood from a damaged site may cover the site to be sutured again. Accordingly, proper suturing of a damaged bleeding site requires a high degree of skill of a doctor.

### SUMMARY OF THE INVENTION

An object of the present invention is to provide a hemostatic device that is capable of stanching a damaged bleeding site easily and quickly.

To this end, the hemostatic device according to the present invention includes a hemostatic head that includes a peripheral end surface formed at a distal end of the hemostatic head, the peripheral end surface extending along an outer edge region of the distal end in a circumferential direction of the hemostatic head, the peripheral end surface having suction ports formed on the peripheral end surface, a recess that is open at the distal end and that is surrounded by the peripheral end surface and a plurality of suction channels that are formed inside the hemostatic head, the suction channels communicating with the respective suction ports. The device also includes a suction device for applying suction pressure to the suction ports via the suction channels, the suction device being coupled to the suction channels.

The present invention can provide a hemostatic device that is capable of stanching a damaged bleeding site easily and quickly.

The above and other objects, features and advantages of the present invention will become apparent from the following description with reference to the accompanying drawings which illustrate examples of the present invention.

### BRIEF DESCRIPTION OF THE DRAWINGS

FIGS. 1A and 1B illustrate a hemostatic device according to a first embodiment of the present invention;

FIG. 2 is a perspective view of the cup portion of a hemostatic head shown in FIG. 1A as viewed obliquely from below;

FIG. 3 is a sectional view of the cup portion of the hemostatic head that is applied to the damaged site of a patient;

FIGS. 4A and 4B illustrate a first variation of the hemostatic head shown in FIGS. 1A to 3;

FIGS. 5A and 5B illustrate a second variation of the hemostatic head shown in FIGS. 1A to 3;

FIGS. 6A and 6B illustrate a variation (third variation) of the example shown in FIG. 5C;

FIGS. 7A and 7B illustrate a variation of the example shown in FIGS. 6A and 6B;

FIG. 8 is a sectional view of a fourth variation of the hemostatic head shown in FIGS. 1A to 3;

FIGS. 9A and 9B illustrate a fifth variation of the hemostatic head shown in FIGS. 1A to 3;

FIGS. 10A and 10B illustrate a sixth variation of the hemostatic head shown in FIGS. 1A to 3;

FIG. 11 is a schematic view showing the configuration of a hemostatic device according to a second embodiment of the present invention;

FIGS. 12A and 12B are diagrams illustrating a method of stanching the bleeding damaged site of a patient by means of the hemostatic device shown in FIG. 11;

FIG. 13 is a schematic view showing a first variation of the hemostatic device shown in FIGS. 11 to 12B;

FIGS. 14A and 14B illustrate a second variation of the hemostatic device shown in FIGS. 11 to 12B; and

FIGS. 15A and 15B illustrate a third variation of the hemostatic device shown in FIGS. 11 to 12B.

### DETAILED DESCRIPTION OF THE INVENTION

Embodiments of the present invention will now be described with reference to the drawings.

### First Embodiment

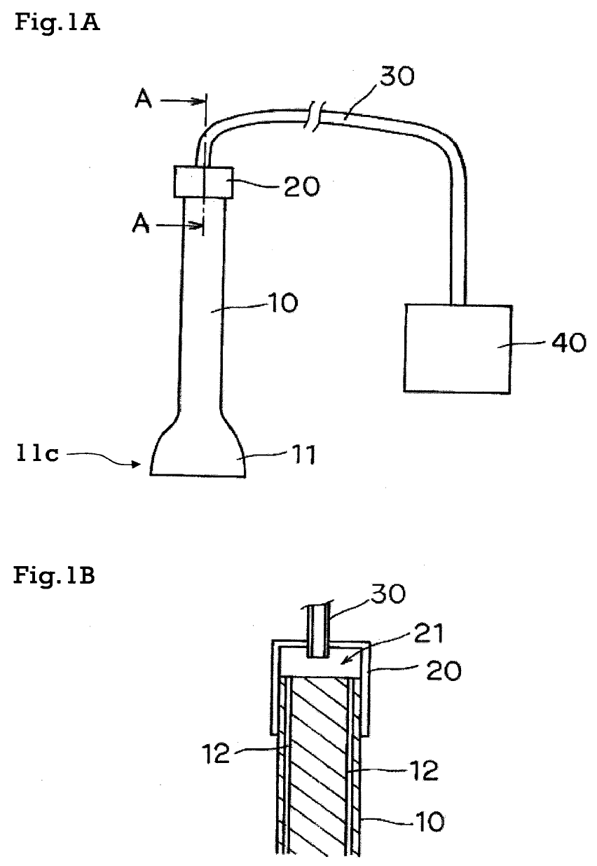

FIGS. 1A and 1B illustrate a hemostatic device according to a first embodiment of the present invention, wherein FIG. 1A is a schematic diagram showing the configuration of a hemostatic device and FIG. 1B is a sectional view taken along line A-A in FIG. 1A.

The hemostatic device shown in FIG. 1A includes elongate hemostatic head 10, suction tube connecting portion 20 attached to a proximal end of hemostatic head 10, suction tube 30 connected to suction tube connecting portion 20, and suction device 40 that applies suction pressure to hemostatic head 10 via suction tube 30. Hemostatic head 10 has, at the distal end thereof, cup portion 11 adapted to be applied to a damaged site, i.e., the bleeding site of a patient.

As shown in FIG. 1B, hemostatic head 10 is provided with a plurality of suction channels 12, which are formed inside hemostatic head 10 and which extend between the distal end and the proximal end of hemostatic head 10. Suction channels 12 are arranged near the outer surface of hemostatic head 10. Space 21 covering the proximal end of hemostatic head 10 is formed inside suction tube connecting portion 20, and suction channels 12 communicate with space 21 at the proximal end of hemostatic head 10. Suction tube 30 connected to suction tube connecting portion 20 also communicates with space 21 at one end of suction tube 30. Thus, by actuating suction device 40, suction pressure is introduced in space 21 inside suction tube connecting portion 20 via suction tube 30, and the suction pressure is also introduced in each suction channel 12 that communicates with space 21.

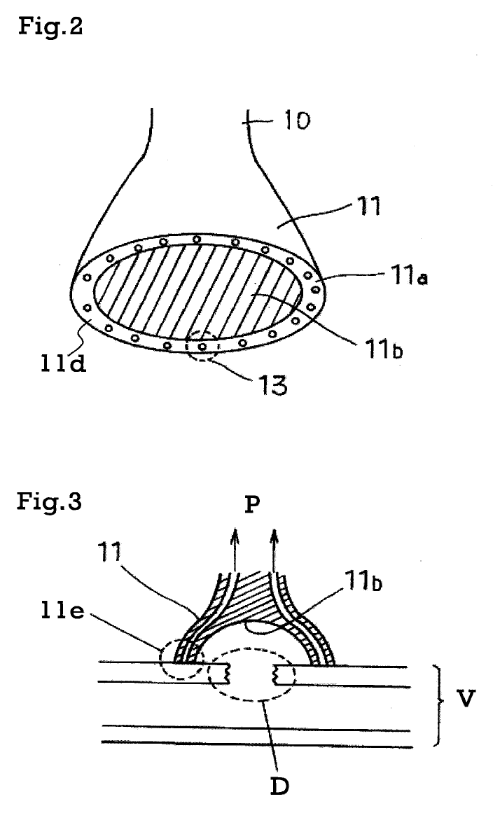

FIG. 2 is a perspective view of the cup portion of the hemostatic head shown in FIGS. 1A and 1B, as viewed obliquely from below. FIG. 3 is a sectional view of the cup portion of the hemostatic head that is applied to the damaged site of a patient.

As shown in FIG. 2, a plurality of suction ports 13 serving as opening ends of suction channels 12 are formed on peripheral end surface 11a that is formed at the distal end 11c of cup portion 11 of hemostatic head 10. Peripheral end surface 11a extends along outer edge region 11d of distal end 11c in the circumferential direction of hemostatic head 10. Suction ports 13 are arranged on peripheral end surface 11a at the same interval. Dome-like recess 11b extending inward within cup portion 11 is formed in the central area of cup portion 11 that is formed at the distal end and that is surrounded by peripheral end surface 11a. When cup portion 11 of hemostatic head 10 is applied to the damaged site of a patient, recess 11b forms a closed space between cup portion 11 and the damaged site.

With reference to FIGS. 1A to 3, a method of stanching blood from the damaged site of a patient by means of the hemostatic device according to the present embodiment will now be described.

First, suction device 40 is actuated to suck in ambient air through suction ports 13 arranged on peripheral edge surface 11a of cup portion 11. Then, cup portion 11, or sucking portion 11e thereof, is pressed against damaged site D of a patient (the damaged site of vessel V, such as an aorta or a vena cava, in the case of FIG. 3) so that the damaged site of a patient is covered with recess 11b that is formed at the distal end of cup portion 11.

Peripheral edge surface 11a arranged at the distal end of cup portion 11 then comes into tight contact with the damaged site of a patient with the aid of suction pressure P applied from a plurality of suction ports 13 that are formed on peripheral end surface 11a. A closed space covering the damaged site is formed by recess 11b of cup portion 11. Since peripheral end surface 11a is in tight contact with and sticks to the damaged site, blood from the damaged site is prevented from flowing out of cup portion 11, even if the blood fills the closed space formed by recess 11b. In this manner, the hemostatic device according to the present embodiment facilitates stanching blood from the damaged site of a patient.

In addition, it is possible to prepare a suturing instrument used to suture the damaged site, as well as to prepare a biomaterial to be applied to the damaged site for the purpose of stanching or conglutination to tissue, while the blood from the damaged site is temporarily stanched by the hemostatic device according to the present embodiment. This prevents the patient from lapsing into a shock state that may be caused by bleeding, which may occur until the instrument and the biomaterial are prepared.

### [First Variation]

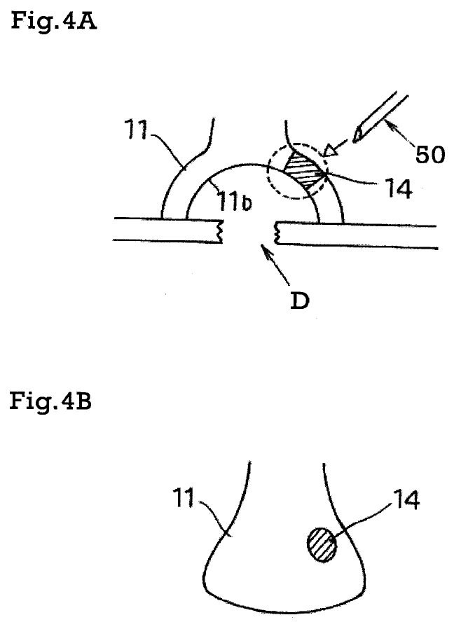

FIGS. 4A and 4B illustrate a first variation of the hemostatic head shown in FIGS. 1A to 3, wherein FIG. 4A is a sectional view of the cup portion formed at the distal end of the hemostatic head, and FIG. 4B is a perspective view of the cup portion of the hemostatic head.

As shown in FIGS. 4A and 4B, the hemostatic head according to the first variation includes, on the side wall of cup portion 11, puncturing port 14, into which injection needle 50 can be inserted. Puncturing port 14 is formed of rubber material, such as silicone rubber, or soft resin material. Puncturing port 14 can be made, for example, by forming an opening on the side wall of cup portion 11 and by filling the opening with rubber, resin material or the like by means of a molding technique, such as insert molding.

The other configurations of the hemostatic device according to this variation are similar to those of the first embodiment that is described above with reference to FIGS. 1A to 3, and detailed descriptions are omitted.

According to the hemostatic head according to the present variation, blood from the damaged site can be stanched by pressing cup portion 11 against the damaged site of a patient, as described above. In addition, an injection needle can be inserted into the closed space of cup portion 11, which is formed by recess 11b, via puncturing port 14 so that a hemostatic agent is administered to the damaged site. Thus, the hemostatic head according to the present variation makes it possible to stanch the bleeding site with an agent while the damaged site is temporarily stanched.

### [Second Variation]

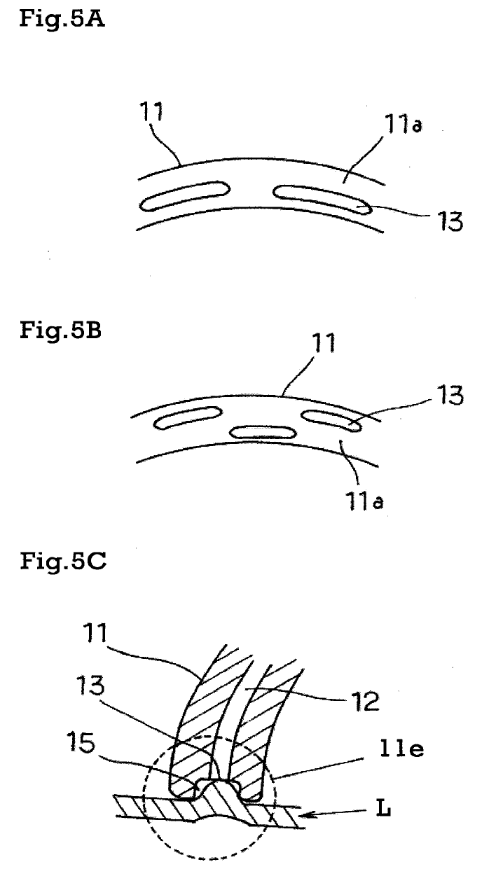

FIGS. 5A to 5C illustrate a second variation of the hemostatic head shown in FIGS. 1A to 3, wherein FIG. 5A is a partial enlarged view of the peripheral end surface of the cup portion of a hemostatic head, FIG. 5B is a partial enlarged view of the peripheral end surface of the cup portion of another hemostatic head and FIG. 5C is a partial enlarged sectional view of yet another hemostatic head, illustrating a portion including the peripheral end surface of the cup portion.

In the variation shown in FIG. 5A, each suction port 13 is formed in a slot shape extending along the circumferential direction of cup portion 11. This configuration ensures more tight contact of peripheral end surface 11a of cup portion 11 with living tissue around the damaged site of a patient because each of suction ports 13 has an increased sucking area.

Moreover, as shown in FIG. 5B, suction ports 13 adjacent to each other may be arranged at radially different distances measured from the center of cup portion 11. This configuration makes it possible to arrange a plurality of suction ports 13 more closely to each other on peripheral end surface 11a, enabling more tight contact of peripheral end surface 11a of cup portion 11 with living tissue.

Furthermore, as shown in FIG. 5C, recessed portion or groove 15 having a larger opening area than suction port 13 may be formed around the periphery of each suction port 13. Groove 15 may be formed either around the periphery of slot-shaped suction port 13 shown in FIGS. 5A to 5B or around the periphery of round-shaped suction port 13 shown in FIG. 2. This configuration allows living tissue L, sucked by suction port 13, to enter recessed portion 15, thereby increasing the sucking area for the living tissue, as compared to the above-described configuration. Accordingly, tighter contact of peripheral end surface 11a of cup portion 11 with living tissue can be ensured.

### [Third Variation]

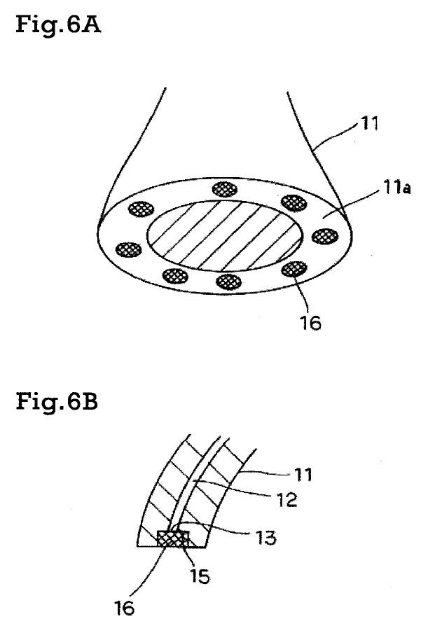

FIGS. 6A and 6B illustrate a further variation (third variation) of the example shown in FIG. 5C, wherein FIG. 6A is a perspective view of the cup portion of the hemostatic head, as viewed obliquely from below and FIG. 6B is an enlarged sectional view of the cup portion, illustrating a portion including the peripheral end surface of the cup portion.

In the configuration shown in FIGS. 6A and 6B, sponge member 16 is provided in recessed portion 15 that is formed around the periphery of each suction port 13. The configuration shown in FIG. 5C may allow blood or the like to be sucked into suction channels 12. However, sponge member 16 located around the periphery of suction port 13, as shown in FIGS. 6A and 6B, prevents blood or the like from being sucked into suction channels 12.

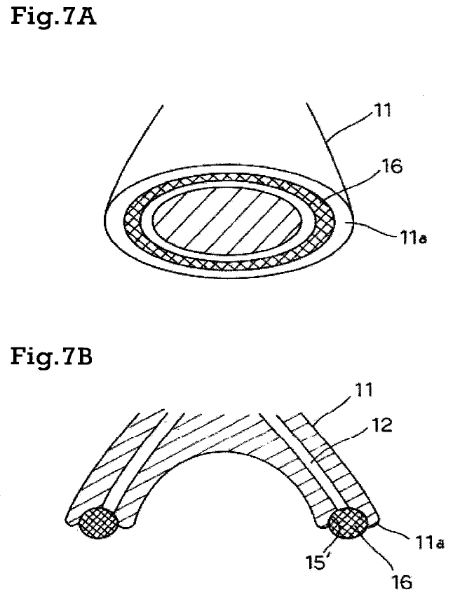

FIGS. 7A and 7B illustrate a variation of the example shown in FIGS. 6A and 6B, wherein FIG. 7A is a perspective view of the cup portion of the hemostatic head, as viewed obliquely from below and FIG. 7B is a sectional view of the cup portion of the hemostatic head.

In the configuration shown in FIGS. 7A and 7B, annular groove 15′ extends through the plurality of suction ports 13 on and along peripheral end surface 11a of cup portion 11 in the circumferential direction. Annular sponge member 16 is provided in groove 15′. This configuration, as well as the example shown in FIGS. 6A and 6B, prevents blood or the like from being sucked into suction channels 12 because the sponge member 16 is located around the periphery of suction ports 13. The configuration of FIGS. 7A and 7B can be obtained by forming one annular groove 15′ and by placing one sponge member 16 therein. Accordingly, this configuration shown in FIGS. 7A and 7B is easier to manufacture than the configuration of FIGS. 6A and 6B, in which recessed portions 15 are individually formed around the periphery of respective suction ports 13 and sponge members 16 are arranged in respective suction ports 13.

### [Fourth Variation]

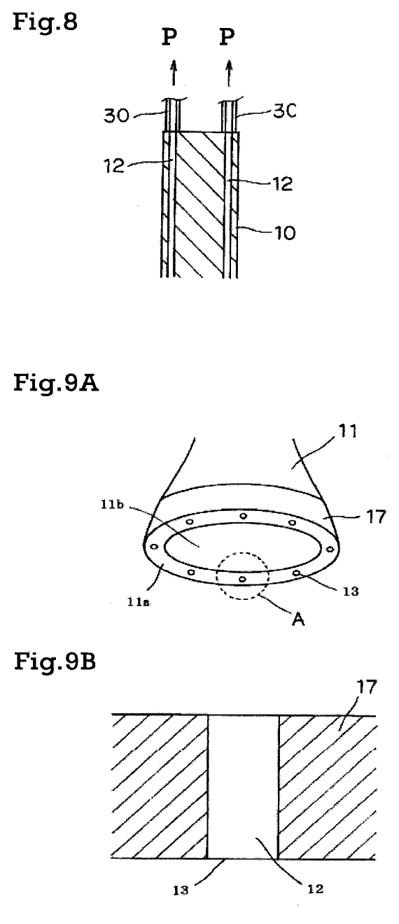

FIG. 8 is a sectional view of a fourth variation of the hemostatic head shown in FIGS. 1A to 3.

In the example shown in FIG. 8, suction tubes 30 are individually coupled to respective suction channels 12 in hemostatic head 10, and suction devices, not shown, are individually coupled to respective suction tubes 30. In other words, suction tube 30 and suction device are provided for each suction channel 12.

In the configuration of FIG. 1A, suction pressure is applied from common suction tube 30 to all suction channels 12 via suction tube connecting portion 20. If one of suction ports 13 remains open without coming into contact with the living tissue, then the suction pressure decreases. This may reduce the sticking force that other suction ports 13 exert on the living tissue. However, even if one of suction ports 13 remains open in the configuration of FIG. 8 in which suction tube 30 and a suction device are provided for each suction channel 12, the sucking function of other suction ports 13 is not affected. Consequently, the living tissue can be stably sucked in.

### [Fifth Variation]

FIGS. 9A and 9B illustrate a fifth variation of the hemostatic head shown in FIGS. 1A to 3, wherein FIG. 9A is a perspective view of the cup portion of the hemostatic head, as viewed obliquely from below and FIG. 9B is an enlarged sectional view of portion A of FIG. 9A.

In the example shown in FIGS. 9A and 9B, a portion of hemostatic head 10, which includes peripheral end surface 11a at the distal end thereof, is formed of elastic member 17 made up of elastic material, such as silicone rubber. Suction channels 12 extend through elastic member 17. A plurality of suction ports 13 are formed on peripheral end surface 11a formed at the distal end of elastic member 17.

Elastic member 17 can be deformed along the surface of the living tissue due to the elastic deformability thereof when it is pressed against the living tissue, ensuring more tight contact of the distal end of cup portion 11 with the living tissue.

### [Sixth Variation]

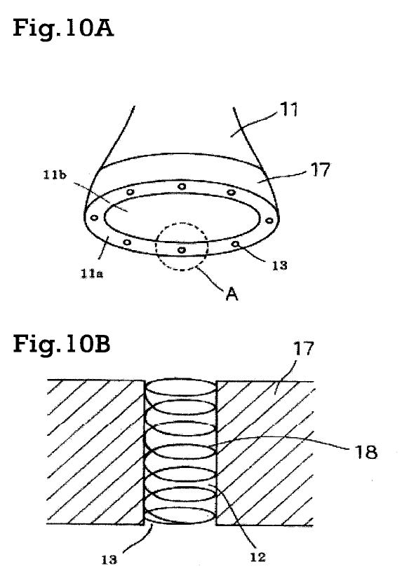

FIGS. 10A and 10B illustrate a sixth variation of the hemostatic head shown in FIGS. 1A to 3, wherein FIG. 10A is a perspective view of the cup portion of the hemostatic head, as viewed obliquely from below and FIG. 10B is an enlarged sectional view of portion A of FIG. 10A.

In the configuration shown in FIGS. 10A and 10B, each suction channel 12 includes, along a portion thereof that extends through elastic member 17, channel deformation preventing member 18 for preventing deformation of suction channel 12. The other configurations are similar to the configuration shown in FIGS. 9A and 9B. Channel deformation preventing member 18 may be, for example, a spiral spring made of metal or hard resin or may be a plurality of rings also made of metal or hard resin.

Deformation of elastic member 17 may deform suction channels 12 and may close suction channels 12 at the deformed portion thereof. Closure of suction channels 12 is not desirable because it may reduce the suction pressure exerted on the living tissue. However, in the configuration shown in FIGS. 10A and 10B, channel deformation preventing member 18, which is provided in each suction channel 12 along a portion thereof that extends through elastic member 17, prevents suction channel 12 from being closed even if elastic member 17 is deformed.

### Second Embodiment

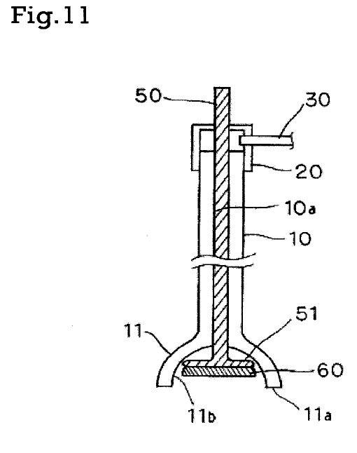

FIG. 11 schematically illustrates the configuration of a hemostatic device according to a second embodiment of the present invention.

Similar to the first embodiment, the hemostatic device shown in FIG. 11 includes hemostatic head 10 provided with cup portion 11 formed at the distal end thereof, suction tube connecting portion 20 attached to the proximal end of hemostatic head 10, suction tube 30 connected to suction tube connecting portion 20 and a suction device, not shown, that applies suction pressure to hemostatic head 10 via suction tube 30. In FIG. 11, a plurality of suction channels formed in hemostatic head 10 are omitted.

Hemostatic head 10 according to the present embodiment includes, in the central portion thereof, through-hole 10a that extends in the longitudinal direction. Pressing member 50 is arranged in through-hole 10a. Pressing member 50 includes, at the distal end thereof, holding portion 51 for holding biomaterial 60. Pressing member 50 extends through the top wall of suction tube connecting portion 20 up to above suction tube connecting portion 20. In a normal condition, holding portion 51 that holds biomaterial 60 is accommodated in a space that is formed by recess 11b of cup portion 11. Biomedical glue is partially applied to the surface of holding portion 51 that holds biomaterial 60 in an amount sufficient for holding biomaterial 60 and for preventing biomaterial 60 from peeling off. The biomedical glue is applied to almost the entire surface of biomaterial 60 which will come into contact with the damaged site of a patient.

Pressing member 50 is slidably arranged in through-hole 10a substantially without a gap between pressing member 50 and through-hole 10a. Similarly, pressing member 50 is slidably inserted into an opening formed through the top wall of suction tube connecting portion 20 substantially without a gap between pressing member 50 and the opening. Thus, when suction pressure is applied to the space in suction tube connecting portion 20 via suction tube 30, substantially no air is allowed to enter the space through the gap between pressing member 50 and through-hole 10a or through the gap between pressing member 50 and the opening on the top wall of suction tube connecting portion 20. If a small amount of air is introduced at all, the air does not affect the suction pressure.

In the present embodiment, suction tube 30 is coupled to suction tube connecting portion 20 at the side surface thereof in order to avoid interference with pressing member 50 that extends upwards above suction tube connecting portion 20.

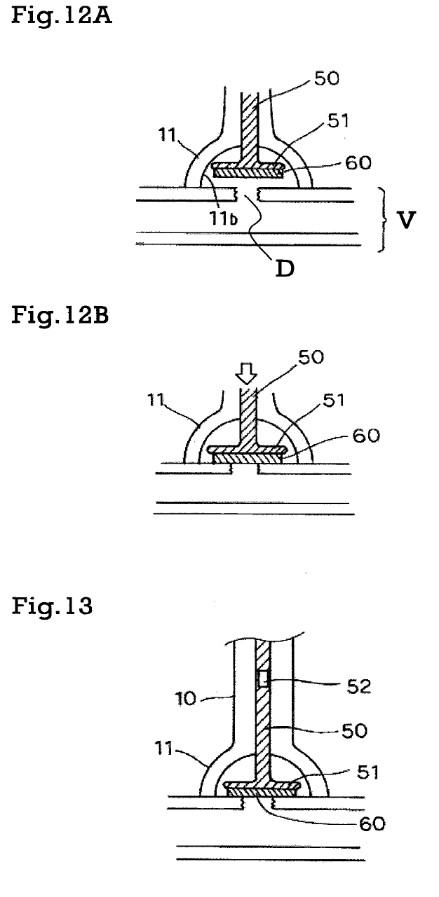

With reference to FIGS. 11, 12A, and 12B, a method of stanching blood from the damaged site of a patient by means of the hemostatic device according to the present embodiment will now be described.

First, suction device, not shown, is actuated to suck in-ambient air through suction ports, not shown, arranged on distal edge surface 11a of cup portion 11. Then, cup portion 11 is pressed against damaged site D of a patient (a damaged site of a vessel V, such as an aorta or a vena cava, in the case of FIGS. 12A and 12B) so that the damaged site of a patient is covered with recess 11b that is formed at the distal end of cup portion 11.

As shown in FIG. 12A, peripheral end surface 11a arranged at the distal end of cup portion 11 then comes into tight contact with the damaged site of a patient with the aid of suction pressure applied from a plurality of suction ports, not shown, that are formed on peripheral end surface 11a. A closed space covering the damaged site is formed by recess 11b of cup portion 11. Since peripheral end surface 11a is in tight contact with and sticks to the damaged site, blood from the damaged site is prevented from flowing out of cup portion 11, even if the blood fills the closed space formed by recess 11b. In this manner, the hemostatic device according to the present embodiment facilitates stanching blood from the damaged site of a patient. The operations described above are similar to the operations using the hemostatic device according to the first embodiment.

In addition, the hemostatic device according to the present embodiment can apply biomaterial 60, which is held by holding portion 51 of pressing member 50, to the damaged site by pressing member 50 downward while the blood from the damaged site is temporarily stanched. As described above, biomedical glue is applied to the surface of biomaterial 60 that will come into contact with the damaged site of a patient, and applied in an area that is larger than the opposite surface of biomaterial 60 that is in contact with holding portion 51. Thus, biomaterial 60 can be applied to the damaged site by pressing biomaterial 60 against the damaged site by means of pressing member 50 and then by detaching pressing member 50 from biomaterial 60 because a larger adhering force is exerted on the damaged site compared to the force exerted on holding portion 51. Biomaterial 60 has a function of repairing the defective site in cooperation with cells that proliferate at the defective site.

Thus, the present embodiment enables not only temporarily stanching blood from the damaged site but also performing hemostatic treatment by adhering biomaterial 60 to the defective site. Therefore, hemostatic treatment can be performed more quickly on the damaged site.

### [First Variation]

FIG. 13 schematically illustrates the configuration of a first variation of the hemostatic device shown in FIGS. 11 to 12B.

In the variation shown in FIG. 13, vibration element 52 for generating ultrasonic vibration is provided inside pressing member 50. Vibration element 52 can be energized through electric wire, not shown, that is connected to vibration element 52. The other configurations of the present variation are similar to those of the hemostatic device according to the second embodiment shown in FIGS. 11 to 12B.

According to the present variation, vibration element 52 is driven while biomaterial 60 is pressed against the damaged site of a patient by means of pressing member 50, as shown in FIG. 13, and improved conglutination of biomaterial 60 to the damaged site can be ensured.

### [Second Variation]

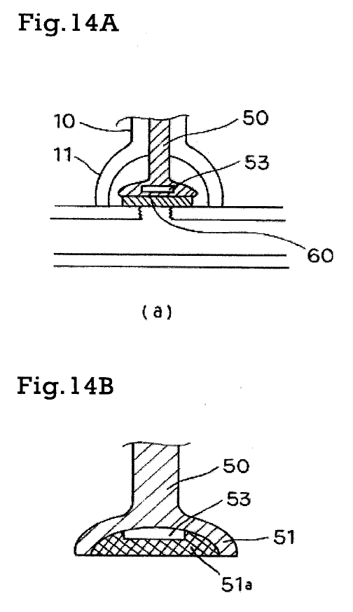

FIGS. 14A and 14B illustrate a second variation of the hemostatic device shown in FIGS. 11 to 12B, wherein FIG. 14A is a sectional view of the distal end portion of the hemostatic head and FIG. 14B is an enlarged sectional view of the distal end portion of the pressing member shown in FIG. 14A.

In the variation shown in FIGS. 14A and 14B, heater 53 for heating holding portion 51 is provided in holding portion 51 of pressing member 50. Heater 53 can be energized through an electric wire, not shown, that is connected to heater 53. Heat conducting portion 51a is provided at the distal end of holding portion 51. Heat conducting portion 51a surrounds heater 53 and forms a surface to which biomaterial 60 is attached. Heat conducting portion 51a is formed of a material having higher heat conductivity than the material that constitutes pressing member 50. The other configurations of the present variation are similar to those of the hemostatic device according to the second embodiment shown in FIGS. 11 to 12B.

According to the present variation, biomaterial 60 is heated via heat conducting portion 51a that is heated by heater 53 while biomaterial 60 is pressed against the damaged site of a patient by means of pressing member 50, as shown in FIG. 14A. Accordingly, improved conglutination of biomaterial 60 to the damaged site can be ensured.

### [Third Variation]

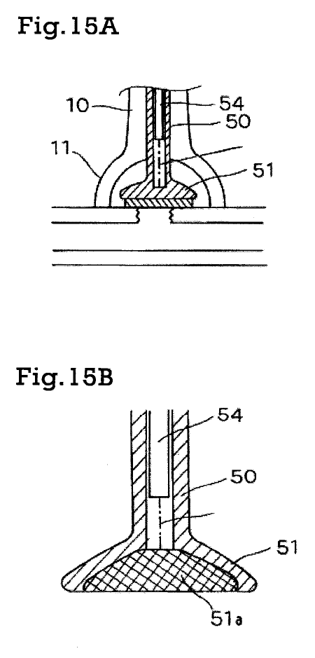

FIGS. 15A and 15B illustrate a third variation of the hemostatic device shown in FIGS. 11 to 12B, wherein FIG. 15A is a sectional view of the distal end portion of the hemostatic head and FIG. 15B is an enlarged sectional view of the distal end portion of the pressing member shown in FIG. 15A.

In the variation shown in FIGS. 15A and 15B, laser emitting portion 54 for emitting laser light to heat holding portion 51 is provided in pressing member 50. Heat conducting portion 51a is provided at the distal end of holding portion 51 and forms a surface to which biomaterial 60 is attached. Laser light emitted from laser emitting portion 54 is adapted to be incident on heat conducting portion 51a. Heat conducting portion 51a is formed of a material having higher heat conductivity than the material that constitutes pressing member 50. The other configurations of the present variation are similar to those of the hemostatic device according to the second embodiment shown in FIGS. 11 to 12B.

According to the present variation, biomaterial 60 is heated by heat conducting portion 51a, which is heated with laser light emitted from laser emitting portion 54, while biomaterial 60 is pressed against the damaged site of the patient by means of pressing member 50, as shown in FIG. 15A. Accordingly, improved conglutination of biomaterial 60 to the damaged site can be ensured.

Whereas various embodiments and variations of the hemostatic device according to the present invention have been described, it should be noted that the hemostatic device according to the present invention is not limited to the above-described embodiments and variations. The configurations according to the above-described embodiments and the variations can be combined with each other where possible. It should also be understood that various changes and modifications may be made without departing from the spirit or scope of the appended claims.

What is claimed is:

1. A hemostatic device comprising:
a hemostatic head including a peripheral end surface formed at a distal end of the hemostatic head, the peripheral end surface extending along an outer edge region of the distal end in a circumferential direction of the hemostatic head, the peripheral end surface having suction ports formed on the peripheral end surface, a recess that is open at the distal end and that is surrounded by the peripheral end surface and a plurality of suction channels that are formed inside the hemostatic head, the suction channels communicating with the respective suction ports; and
a suction device for applying suction pressure to the suction ports via the suction channels, the suction device being coupled to the suction channels.

2. The hemostatic device according to claim 1, wherein a puncturing port is provided on the hemostatic head, wherein an injection needle can be inserted from outside of the hemostatic head into a space that is formed by the recess.

3. The hemostatic device according to claim 1, wherein the suction ports are arranged at a same interval.

4. The hemostatic device according to claim 1, wherein each suction port is formed in a slot-like shape that extends along the circumferential direction.

5. The hemostatic device according to claim 1, wherein the suction ports adjacent to each other are arranged at different distances measured from a center of the peripheral end surface.

6. The hemostatic device according to claim 1, wherein a recessed portion is formed around each suction port, the recessed portion having a larger opening area than the corresponding suction port.

7. The hemostatic device according to claim 6, wherein a sponge member is provided in each recessed portion.

8. The hemostatic device according to claim 1, wherein an annular groove is formed on the peripheral end surface, the annular groove extending through the suction ports, wherein an annular sponge member is provided in the annular groove.

9. The hemostatic device according to claim 1, wherein a portion of the hemostatic head which includes the peripheral end surface is formed of an elastic member.

10. The hemostatic device according to claim 9, wherein each suction channel is provided with a channel deformation preventing member for preventing deformation of the suction channel, wherein the channel deformation preventing member is located along a portion of the suction channel, the portion extending through the elastic member.

11. The hemostatic device according to claim 1, further including
a through-hole formed in the hemostatic device, the through-hole communicating with the recess, and
a pressing member that is arranged in the through-hole, wherein the pressing member has a holding portion formed at a distal end thereof, the holding portion being adapted to hold biomaterial while the biomaterial is accommodated in a space formed by the recess.

12. The hemostatic device according to claim 11, wherein the pressing member includes a vibration element for generating ultrasonic vibration.

13. The hemostatic device according to claim 11, wherein the pressing member includes a heater for heating the holding portion.

14. The hemostatic device according to claim 11, wherein the pressing member includes a laser emitting portion that irradiates the holding portion with laser light in order to heat the holding portion.

15. The hemostatic device according to claim 1, wherein each suction device is individually coupled to each suction channel such that the suction pressure is individually applied to each suction channel.

16. A method to stanch a damaged site comprising:
pressing a hemostatic head against a damaged site, the hemostatic head including a peripheral end surface formed at a distal end of the hemostatic head, the peripheral end surface extending along an outer edge region of the distal end in a circumferential direction of the hemostatic head, the peripheral end surface having suction ports formed on the peripheral end surface and a recess that is open at the distal end and that is surrounded by the peripheral end surface, wherein the hemostatic head is pressed against the damaged site while ambient air is sucked in by the suction ports, the hemostatic head being pressed such that the damaged site is surrounded by the peripheral end surface and is accommodated within the recess.

17. The method to stanch a damaged site according to claim 16, further comprising applying a biomaterial to the damaged site while the hemostatic head is pressed against the damaged site, the biomaterial being accommodated in the recess in advance.
